(19) United States
(12) Patent Application Publication
(10) Pub. No.: US 2025/0044361 A1
(43) Pub. Date: Feb. 6, 2025

(54) METHOD FOR UPDATING STATE OF CHARGE BASED ON POWER CHARACTERISTIC OF ELECTROCHEMICAL MODEL OF LITHIUM-ION BATTERY

(71) Applicant: TSINGHUA UNIVERSITY, Beijing (CN)

(72) Inventors: Qixin CHEN, Beijing (CN); Yuanbo CHEN, Beijing (CN); Yuxuan GU, Beijing (CN); Hongye GUO, Beijing (CN); Kedi ZHENG, Beijing (CN); Ruike LYU, Beijing (CN)

(21) Appl. No.: 18/919,638

(22) Filed: Oct. 18, 2024

Related U.S. Application Data
(63) Continuation of application No. PCT/CN2022/091037, filed on May 5, 2022.

(30) Foreign Application Priority Data
Apr. 22, 2022 (CN) 202210431423.3

(51) Int. Cl. G01R 31/367; G01R 31/378; G01R 31/3842

(52) U.S. Cl. CPC G01R 31/367; G01R 31/378; G01R 31/3842

(57) ABSTRACT

A method for updating a state of charge based on power characteristics of an electrochemical model of a lithium-ion battery includes obtaining an initial state of charge of a battery and a current sequence with a constant amplitude; obtaining a port voltage of the battery at each moment within a preset time period based on the initial state information about the battery and electrochemical model simulation of the lithium-ion battery; obtaining a current amplitude-state of charge-port power curved surface based on the port powers; fitting the current amplitude-state of charge-port power curved surface to a plane equation; and obtaining a current amplitude corresponding to the port power and the state of charge by using the plane equation based on the port power and the state of charge, and updating the state of charge based on the current amplitude and the preset time period.

### CROSS-REFERENCE TO RELATED APPLICATIONS

This application is a Continuation Application of International Application PCT/CN2022/091037, filed May 5, 2022, which claims priority to Chinese Patent Application No. 202210431423.3, filed on Apr. 22, 2022, the entire disclosures of which are hereby incorporated herein by reference.

### FIELD

The present disclosure relates to the field of energy management and operation technology of a lithium-ion battery, and particularly to a method and a device for updating a state of charge based on power characteristics of an electrochemical model of a lithium-ion battery.

### BACKGROUND

As a flexible, efficient and safe energy storage medium, a lithium-ion battery is widely used in power, traffic and other business fields. In a process of energy management and operation of the lithium-ion battery, the state of charge of a battery is one of key information concerned by managers. In order to obtain an accurate description of a state of charge to meet a requirement of efficient operation of the lithium-ion battery, a time-varying port voltage characteristic of the battery needs to be considered using an electrochemical model. In a process of characterizing the time-varying port characteristic, not only should its accuracy be guaranteed, but also computational efficiency for updating the state of charge should be considered in practical applications.

At present, the lithium-ion battery models may be divided into four categories in engineering applications: a source & sink model, an equivalent circuit model, a data-driven black-box model, an electrochemical model based on chemical reaction mechanism. The source & sink model and the equivalent circuit model are most widely used in engineering, and battery voltage is determined or measured by a manufacturer in a process of updating the state of charge, and is regarded as a constant. For example, scholars from Massachusetts Institute of Technology and Argon National Laboratory use the equivalent circuit model in case of studying arbitrage capacity of the battery in a microgrid, regard the battery voltage as a constant value, and use port power to directly update the state of charge. Regarding the electrochemical model and the black-box model, the state of charge thereof is generally obtained in a continuous time sequence simulation using a state estimation method, for example, scholars from Beijing University of Technology and University of Michigan determine the current state of charge of the battery in the electrochemical model using a joint state estimation method. However, taking the port voltage of the battery as the constant value essentially ignores a change of the port voltage relative to the state of charge of the battery during operation of the battery, resulting in a large error in the process of updating the state of charge. Moreover, in case that the state estimation method in the continuous time sequence simulation is used to determine the state of charge, it is necessary to repeatedly solve a nonlinear high-order differential state equation in the electrochemical model, which requires a lot of computational resources and has low computational efficiency, and is difficult to adapt to the energy management and operation of the battery in a long period of time.

### SUMMARY

Embodiments of a first aspect of the present disclosure provide a method for updating a state of charge based on power characteristics of an electrochemical model of a lithium-ion battery, comprising: S1: obtaining an initial state of charge of a battery and a current sequence with a constant amplitude; S2: obtaining initial state information about the battery, and obtaining a port voltage of the battery at each moment within a preset time period based on the initial state information about the battery and electrochemical model simulation of the lithium-ion battery; S3: calculating port power corresponding to the initial state of charge based on the port voltage of the battery and the amplitude of the current sequence; S4: adjusting the initial state of charge and the amplitude of the current sequence, repeating steps S1-S3 to respectively obtain port powers corresponding to a plurality of different initial states of charge and a plurality of different amplitudes of the current sequence, and obtaining a current amplitude-state of charge-port power curved surface based on the port powers; S5: fitting the current amplitude-state of charge-port power curved surface to a plane equation; and S6: obtaining a current amplitude corresponding to the port power and the state of charge by using the plane equation based on the port power and the state of charge, and updating the state of charge based on the current amplitude and the preset time period.

Embodiments of a second aspect of the present disclosure provide an electronic device, comprising: a memory; a processor; and a computer program stored in the memory and executable on the processor, wherein the computer program, when executed by the processor, implements the above-mentioned method for updating the state of charge based on power characteristics of the electrochemical model of the lithium-ion battery.

Embodiments of a third aspect of the present disclosure provide a non-transitory computer readable storage medium having stored thereon a computer program, wherein the computer program, when executed by a processor, implements the above-mentioned method for updating the state of charge based on power characteristics of the electrochemical model of the lithium-ion battery.

Embodiments of a fourth aspect of the present disclosure provide a computer program product, comprising a computer program, wherein the computer program, when executed by a processor, implements the above-mentioned method for updating the state of charge based on power characteristics of the electrochemical model of the lithium-ion battery.

Additional aspects and advantages of the present disclosure will be given in part in the following descriptions, become apparent in part from the following descriptions, or be learned from the practice of the present disclosure.

### BRIEF DESCRIPTION OF THE DRAWINGS

These and/or other aspects and advantages of the present disclosure will become apparent and more readily appreciated from the following descriptions of embodiments made with reference to the drawings, in which:

FIG. 1 is a flow chart illustrating a method for updating a state of charge based on power characteristics of an electrochemical model of a lithium-ion battery provided in Embodiment 1 of the present disclosure;

FIG. 2 is a schematic structural diagram illustrating a lithium-ion battery cell based on a method for updating a state of charge based on power characteristics of an electrochemical model of a lithium-ion battery in embodiments of the present disclosure;

FIG. 3 is another flow chart illustrating a method for updating a state of charge based on power characteristics of an electrochemical model of a lithium-ion battery in embodiments of the present disclosure;

FIG. 4 is a schematic block diagram illustrating a device for updating a state of charge based on power characteristics of an electrochemical model of a lithium-ion battery provided in Embodiment 2 of the present disclosure.

### DETAILED DESCRIPTION

Embodiments of the present disclosure will be described in detail and examples of embodiments are illustrated in the drawings. The same or similar elements and the elements having the same or similar functions are denoted by like reference numerals throughout the descriptions. Embodiments described herein with reference to drawings are explanatory, serve to explain the present disclosure, and are not construed to limit the present disclosure.

In existing research on energy operation and management of a lithium-ion battery, (1) in case that port voltage is regarded as a constant value, port power is directly used to determine a state of charge, and it is difficult to ensure accuracy for updating the state of charge; (2) in case that state estimation is carried out in continuous time sequence simulation, computational efficiency is low, and a lot of computational resources are needed in long-term management. In view of the above problems, an efficient method for updating the state of charge that may reflect real power characteristics in battery operation needs to be studied. Therefore, the method for updating the state of charge based on power characteristics of an electrochemical model of the lithium-ion battery, not only needs time-varying characteristic of the port voltage of the battery to be accurately considered, but also calculation efficiency in long-term energy operation and management of the battery needs to be met.

Therefore, the object of the present disclosure is to propose a method for updating a state of charge based on power characteristics of an electrochemical model of a lithium-ion battery, which solves the technical problem of mutual exclusion of precision and efficiency for updating the state of charge of the lithium-ion battery in the existing method, and uses high simulation precision of external characteristics of the electrochemical model of the lithium-ion battery, and takes into consideration the influence of time-varying port voltage of the battery on update of the state of charge under the condition of not increasing the calculation complexity, so that the update of the state of charge of the battery is completed without continuous time sequence simulation under the condition of knowing the power and the state of charge of the battery, the calculation efficiency of the electrochemical model under the power application scene is improved, and the application scene of the electrochemical model in engineering is expanded.

The present disclosure proposes a method for updating the state of charge based on the power characteristics of the electrochemical model of the lithium-ion battery. By utilizing high simulation precision of the external characteristics of the electrochemical model of the lithium-ion battery and taking into account the influence of time-varying port voltage of the battery on updating of the state of charge without increasing computational complexity, the updating of the state of charge of the battery is achieved without performing continuous time sequence simulation in case that the power and the state of charge of the battery are known, which improves computational efficiency of the electrochemical model in power application scenarios and expands the application scenarios of the electrochemical model in engineering.

The related art of the present disclosure includes: construction and simulation technology of the electrochemical model of the lithium-ion battery: a lithium-ion electrochemical model consists of a set of nonlinear high-order differential state equations, which provides more accurate internal state information and external characteristic information by accurately describing the internal chemical reactions of the battery. Multivariate equation fitting technology: multivariate equation fitting is to connect a series of sample points in a three-dimensional or high-dimensional space with a smooth surface so that the surface may approximate distribution of the sample points. In the present disclosure, a plane fitting in a three-dimensional space is used, involving a sample point fitting of three variables, to provide a to-be-determined fitting coefficient.

Embodiments of a first aspect of the present disclosure provide a method for updating a state of charge based on power characteristics of an electrochemical model of a lithium-ion battery, comprising: S1: obtaining an initial state of charge of a battery and a current sequence with a constant amplitude; S2: obtaining initial state information about the battery, and obtaining a port voltage of the battery at each moment within a preset time period based on the initial state information about the battery and electrochemical model simulation of the lithium-ion battery; S3: calculating port power corresponding to the initial state of charge based on the port voltage of the battery and the amplitude of the current sequence; S4: adjusting the initial state of charge and the amplitude of the current sequence, repeating steps S1-S3 to respectively obtain port powers corresponding to a plurality of different initial states of charge and a plurality of different amplitudes of the current sequence, and obtaining a current amplitude-state of charge-port power curved surface based on the port powers; S5: fitting the current amplitude-state of charge-port power curved surface to a plane equation; and S6: obtaining a current amplitude corresponding to the port power and the state of charge by using the plane equation based on the port power and the state of charge, and updating the state of charge based on the current amplitude and the preset time period.

In one embodiment of the present disclosure, the initial state information about the battery comprises: a lithium concentration on a surface of an electrode active material, an average lithium concentration of the electrode active material, a lithium concentration of an electrode electrolyte, and an initial temperature of the battery.

In one embodiment of the present disclosure, obtaining the port voltage of the battery at each moment within the preset time period based on the initial state information about the battery and the electrochemical model simulation of the lithium-ion battery comprises:
obtaining an ambient temperature sequence of the battery with a constant amplitude, wherein the ambient temperature sequence of the battery comprises an ambient temperature of the battery at each moment within the preset time period,
at a starting moment of the preset time period, updating a parameter vector at a current moment based on the lithium concentration of the electrode electrolyte, the average lithium concentration of the electrode active material and the battery temperature at a previous moment:

$$θ ( k + 1 ) = {f}_{θ} ( {c}_{e} ( k ) , {c}_{s , av} ( k ) , {T}_{b} ( k ) )$$

where θ(k+1) represents the parameter vector at the current moment, ƒ_{θ} represents a parameter update function, cₑ(k) represents the lithium concentration of the electrode electrolyte at the previous moment, c_{s,av}(k) represents the average lithium concentration of the electrode active material at the previous moment, and T_{b}(k) represents the battery temperature at the previous moment;
updating a reaction current intensity at the current moment based on the lithium concentration of the electrode electrolyte, the lithium concentration on the surface of the electrode active material, the battery temperature, and port current at the previous moment and the parameter vector at the current moment:

$${j}_{n} ( k + 1 ) = {f}_{j} ( {c}_{e} ( k ) , {c}_{s , surf} ( k ) , {T}_{b} ( k ) , I ( k ) , θ ( k + 1 ) )$$

where jₙ(k+1) represents the reaction current intensity at the current moment, ƒⱼ represents a reaction current update function, c_{s,surf}(k) represents the lithium concentration on the surface of the electrode active material at the previous moment, and I(k) represents the port current at the previous moment;
updating a potential difference on a solid-solution surface of an electrode at the current moment based on the reaction current intensity and the parameter vector at the current moment:

$${ϕ}_{s e} ( k + 1 ) = {f}_{ϕ} ( {j}_{n} ( k + 1 ) , θ ( k + 1 ) )$$

where ϕₛₑ(k+1) represents the potential difference on the solid-solution surface of the electrode at the current moment, and ƒ_{ϕ} represents an update function of the potential difference on the solid-solution surface of the electrode;
updating the lithium concentration of the electrode active material at the current moment based on the average lithium concentration of the electrode active material and the lithium concentration on the surface of the electrode active material at the previous moment, the reaction current intensity at the current moment, the parameter vector at the current moment and a sampling interval:

$${c}_{s , av} ( k + 1 ) = {f}_{a v} ( {c}_{s , av} ( k ) , {c}_{s , surf} ( k ) , {j}_{n} ( k + 1 ) , θ ( k + 1 ) , Δ t )$$

$${c}_{s , surf} ( k + 1 ) = {f}_{surf} ( {c}_{s , av} ( k ) , {c}_{s , surf} ( k ) , {j}_{n} ( k + 1 ) , θ ( k + 1 ) , Δ t )$$

where c_{s,av}(k+1) represents the average lithium concentration of the electrode active material at the current moment, ƒₐᵥ represents an update function of the average lithium concentration of the electrode active material, Δt represents the sampling interval, c_{s,surf}(k+1) represents the lithium concentration on the surface of the electrode active material at the current moment, and ƒ_{surf} represents an update function of the lithium concentration on the surface of the electrode active material;
updating the lithium concentration of the electrode electrolyte at the current moment based on the lithium concentration of the electrode electrolyte and the port current at the previous moment, the parameter vector at the current moment and the sampling interval:

$${c}_{e} ( k + 1 ) = {f}_{e} ( {c}_{e} ( k ) , I ( k ) , θ ( k + 1 ) , Δ t )$$

where cₑ(k+1) represents the lithium concentration of the electrode electrolyte at the current moment, and ƒₑ represents an update function of the lithium concentration of the electrode electrolyte;
obtaining a port voltage V of the battery and a potential difference U in the battery at the current moment based on the lithium concentration of the electrode electrolyte, the lithium concentration on the surface of the electrode active material, the reaction current intensity and the parameter vector at the current moment, and the battery temperature and the port current at the previous moment:

$$V ( k + 1 ) = {f}_{V} ( {c}_{e} ( k + 1 ) , {c}_{s , surf} ( k + 1 ) , {j}_{n} ( k + 1 ) , {T}_{b} ( k ) , I ( k ) , θ ( k + 1 ) )$$

$$U ( k + 1 ) = {f}_{U} ( {c}_{e} ( k + 1 ) , {c}_{s , surf} ( k + 1 ) , {j}_{n} ( k + 1 ) , {T}_{b} ( k ) , I ( k ) , θ ( k + 1 ) )$$

where V(k+1) represents the port voltage of the battery at the current moment, ƒ_{V} represents an update function of the port voltage of the battery, U(k+1) represents the potential difference in the battery at the current moment, and ƒ_{U} represents an update function of the potential difference in the battery;
obtaining the battery temperature at the current moment based on the port voltage of the battery, the potential difference in the battery, the reaction current intensity and the parameter vector at the current moment, and the battery temperature, ambient temperature, the port current and the sampling interval at the previous moment:

$${T}_{b} ( k + 1 ) = {f}_{T} ( V ( k + 1 ) , U ( k + 1 ) , {j}_{n} ( k + 1 ) , {T}_{b} ( k ) , {T}_{amb} ( k ) , I ( k ) , θ ( k + 1 ) , Δ t )$$

where T_{b}(k+1) represents the battery temperature at the current moment, ƒ_{T} represents an update function of the battery temperature, and T_{amb}(k) represents the ambient temperature at the previous moment; and
repeating the above-mentioned simulation, iteration and update steps, and cyclically updating states at the current moment from states at the previous moment: the parameter vector, the reaction current intensity, the potential difference on the solid-solution surface of the electrode, the lithium concentration of the electrode active material, and the lithium concentration of the electrode electrolyte, and outputting the port voltage of the battery and the battery temperature based on a state update result until the preset time period ends to obtain the port voltage of the battery at each moment within the preset time period,
wherein the port voltage of the battery at each moment within the preset time period may be represented as

V=[V₁V₂ . . . Vₖ . . . V_{N}]
where V represents the port voltage of the battery at each moment within the preset time period, Vₖ represents a port voltage of the battery at a k^{th} moment, and N is a total number of moments within the preset time period, wherein N is an integer greater than or equal to 2.

In one embodiment of the present disclosure, calculating the port power corresponding to the initial state of charge based on the port voltage of the battery and the amplitude of the current sequence comprises:
obtaining an average port voltage within the preset time period based on the port voltage of the battery, and calculating the port power corresponding to the initial state of charge and the amplitude of the current sequence based on the average port voltage and the amplitude of the current sequence;
wherein the average port voltage is represented as:

$$\overline{V} = \frac{{V}_{1} + {V}_{2} + … + {V}_{k} + … + {V}_{N}}{N}$$

where V̅ represents the average port voltage; and
the port power is represented as:

$$P ( {SOC}_{0} , {T}_{a m b} ) = I \times \overline{V}$$

where P represents the port power, SOC₀ represents the initial state of charge, T_{amb} represents a set ambient temperature of the battery, and I represents the amplitude of the current sequence.

In one embodiment of the present disclosure, adjusting the initial state of charge and the amplitude of the current sequence, repeating the steps S1-S3 to respectively obtain port powers corresponding to the plurality of different initial states of charge and the plurality of different amplitudes of the current sequence, and obtaining the current amplitude-state of charge-port power curved surface based on the port powers comprise:
adjusting the initial state of charge and the amplitude of the current sequence of the battery, repeating the steps S1-S3 to respectively obtain port powers corresponding to the plurality of different initial states of charge and the plurality of different amplitudes of the current sequence, and smoothly connecting the port powers to obtain the current amplitude-state of charge-port power curved surface under a condition of maintaining the ambient temperature of the battery constant;
wherein the current amplitude-state of charge-port power curved surface is represented as:

$${f}_{P} ( {SOC}_{0} , P , I ) {\vert}_{T = {T}_{a m b}} = 0$$

where ƒ_{P} represents a function of the current amplitude-state of charge-port power curved surface in a case of T=T_{amb}, and T represents an actual ambient temperature of the battery.

In one embodiment of the present disclosure, fitting the current amplitude-state of charge-port power curved surface to the plane equation is represented as:

$${f}_{P} ( {SOC}_{0} , P , I ) {\vert}_{T = {T}_{amb}} \approx A \times {SOC}_{0} + B \times P + C \times I + D = 0$$

where ƒ_{P} represents the function of the current amplitude-state of charge-port power curved surface in the case of T=T_{amb}, A, B and C are first-order coefficients of plane fitting, and D is a constant coefficient of the plane fitting.

In one embodiment of the present disclosure, obtaining the current amplitude corresponding to the port power and the state of charge by using the plane equation based on the port power and the state of charge, and updating the state of charge based on the current amplitude and the preset time period comprise:
obtaining the current amplitude by using the plane equation based on the known port power and state of charge under the condition of maintaining the ambient temperature of the battery constant;
wherein the current amplitude is represented as:

$$I = {f}_{I} ( {SOC}_{0} , P ) {\vert}_{T = {T}_{amb}} \approx {a}_{0} ( {T}_{amb} ) + {a}_{1} ( {T}_{amb} ) \times {SOC}_{0} + {a}_{2} ( {T}_{amb} ) \times P$$

where I represents the current amplitude, ƒ_{I} represents a current amplitude function, a₀ represents a constant coefficient of the current amplitude function, a₁ represents a first-order coefficient corresponding to SOC₀ in the current magnitude function, a₂ represents a first-order coefficient corresponding to P in the current magnitude function;
a₀, a₁, a₂ may be derived from the coefficients A, B, C, D of the plane fitting:

$${a}_{0} = - \frac{D}{C}$$

$${a}_{1} = - \frac{A}{C}$$

$${a}_{2} = - \frac{B}{C} ,$$

and
updating the state of charge based on the current amplitude and the preset time period, which is represented as:

$$Δ SOC = \frac{- I \times Δ T}{{C}_{0}}$$

$$Δ T = N Δ t$$

$${SOC}_{T + 1} = {SOC}_{T} + Δ SOC$$

where ΔSOC represents a change in the state of charge in adjacent time periods, C₀ represents a total capacity of the battery in ampere-hour, ΔT represents a length of the preset time period, SOC_{T+1} represents the state of charge of the battery at start of a next time period, and SOC_{T} represents the state of charge of the battery at start of a present time period.

Embodiments of a second aspect of the present disclosure provide an electronic device, comprising: a memory; a processor; and a computer program stored in the memory and executable on the processor, wherein the computer program, when executed by the processor, implements the above-mentioned method for updating the state of charge based on power characteristics of the electrochemical model of the lithium-ion battery.

Embodiments of a third aspect of the present disclosure provide a non-transitory computer readable storage medium having stored thereon a computer program, wherein the computer program, when executed by a processor, implements the above-mentioned method for updating the state of charge based on power characteristics of the electrochemical model of the lithium-ion battery.

Embodiments of a fourth aspect of the present disclosure provide a computer program product, comprising a computer program, wherein the computer program, when executed by a processor, implements the above-mentioned method for updating the state of charge based on power characteristics of the electrochemical model of the lithium-ion battery.

Hereinafter, the method and the device for updating the state of charge based on the power characteristics of the electrochemical model of the lithium-ion battery in embodiments of the present disclosure will be described with reference to the accompanying drawings.

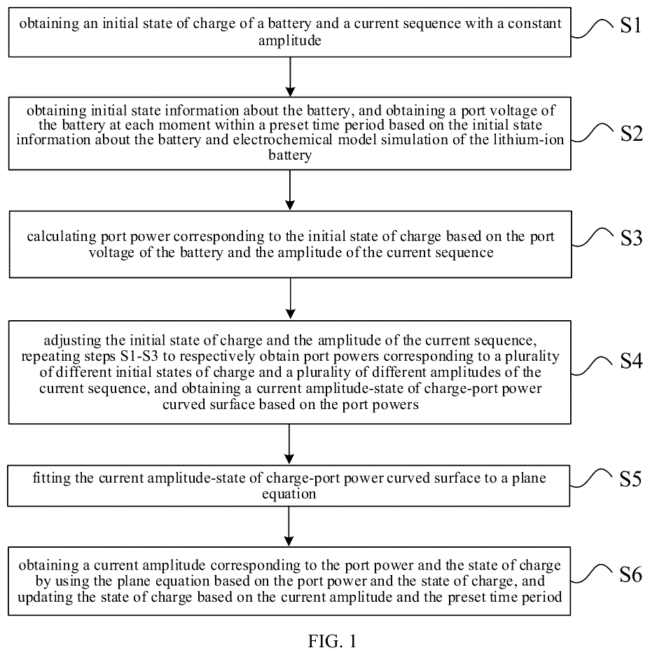

FIG. 1 is a flow chart illustrating a method for updating a state of charge based on power characteristics of an electrochemical model of a lithium-ion battery provided in Embodiment 1 of the present disclosure.

As shown in FIG. 1, the method for updating the state of charge based on the power characteristics of the electrochemical model of the lithium-ion battery includes the following steps:
S1: obtaining an initial state of charge of a battery and a current sequence with a constant amplitude;
S2: obtaining initial state information about the battery, and obtaining a port voltage of the battery at each moment within a preset time period based on the initial state information about the battery and electrochemical model simulation of the lithium-ion battery;
S3: calculating port power corresponding to the initial state of charge based on the port voltage of the battery and the amplitude of the current sequence;
S4: adjusting the initial state of charge and the amplitude of the current sequence, repeating steps S1-S3 to respectively obtain port powers corresponding to a plurality of different initial states of charge and a plurality of different amplitudes of the current sequence, and obtaining a current amplitude-state of charge-port power curved surface based on the port powers;
S5: fitting the current amplitude-state of charge-port power curved surface to a plane equation; and
S6: obtaining a current amplitude corresponding to the port power and the state of charge by using the plane equation based on the port power and the state of charge, and updating the state of charge based on the current amplitude and the preset time period.

With the method for updating the state of charge based on the power characteristics of the electrochemical model of the lithium-ion battery according to embodiments of the present disclosure, the technical problem of mutual exclusion of the precision and efficiency for updating the state of charge of the lithium-ion battery in the existing method is solved. By utilizing high simulation precision of the external characteristics of the electrochemical model of the lithium-ion battery and taking into account the influence of the time-varying port voltage of the battery on updating of the state of charge without increasing computational complexity, the updating of the state of charge of the battery is achieved without performing continuous time sequence simulation in case that the power and the state of charge of the battery are known, which improves computational efficiency of the electrochemical model in power application scenarios and expands the application scenarios of the electrochemical model in engineering.

The present disclosure proposes the method for updating the state of charge based on the power characteristics of the electrochemical model of the lithium-ion battery, where the time-varying characteristic of the port voltage of the lithium-ion battery changing with a change in the state of charge is described by the electrochemical model, and a power-current characteristic description method for constructing the current amplitude-state of charge-port power curved surface and performing the plane fitting is adopted, to avoid performing the long-term continuous time sequence simulation on the electrochemical model in energy management of the battery in a case of improving accuracy of state updating. This method is used to test the battery in a short period of time, and the fitting plane is used to describe power-current characteristics based on the electrochemical model. In a long period of time, the above characteristic plane equation is used to perform relevant calculation of the energy operation and management of the battery.

In the present disclosure, under a condition of retaining the time-varying characteristics of the port voltage during the operation of the lithium-ion battery, without increasing the computational complexity of the energy operation and management of the battery, the electrochemical model of the lithium-ion battery in the present disclosure may be applied to day-ahead planning, joint scheduling and other long-term optimization scenarios without performing continuous time sequence simulation of the electrochemical model with a high computational complexity, which provides the energy operation and management of the battery with information about updating the state of charge which takes into account both accuracy and efficiency, and supports efficient and economic operation of the lithium-ion battery in different scenarios, with important practical significance and good application prospects.

During the scheduling operation of the energy storage system including lithium-ion batteries, the method is used to estimate the state of charge of the lithium-ion batteries in real time, and the scheduling power needs to be adjusted to ensure that the state of charge of the lithium-ion batteries is maintained within the range of 10% to 90%, so as to ensure the performance and appropriate redundancy capacity of the lithium-ion batteries during the operation.

Ambient temperature of the battery is set and represented as T_{amb}. In the present disclosure, the ambient temperature of the battery remains unchanged.

The initial state of charge of the battery is obtained and represented as SOC₀, where a domain of the initial state of charge is [0,1].

The current sequence with the constant amplitude is obtained, which may be represented as:

$$I = {\underbrace{[ \begin{matrix}I & … & I\end{matrix} ]}}_{1 \times N} ,$$

where I represents the amplitude of the current sequence, N represents a total number of moments in the preset time period, a period of action of the current at each moment is represented as tₖ≤t<tₖ₊₁, and the sampling interval is represented as Δt=tₖ₊₁−tₖ.

Further, in embodiments of the present disclosure, the initial state information about the battery includes: a lithium concentration on a surface of an electrode active material, an average lithium concentration of the electrode active material, a lithium concentration of an electrode electrolyte, and an initial temperature of the battery.

Obtaining the initial state information about the battery includes: obtaining a type of an electrode active material used for an electrode to be analyzed, querying an average lithium concentration of the electrode active material corresponding to maximum and minimum states of charge of the electrode active material, obtaining an initial value of the average lithium concentration of the electrode active material according to a direct proportion relationship between the state of charge and the average lithium concentration, setting an initial uniform distribution of the lithium concentration of the electrode active material in an initial state, obtaining that a surface lithium concentration of the electrode active material is equal to the initial value of the average lithium concentration, obtaining an initial value of the lithium concentration of an electrode electrolyte according to parameter settings, and setting an initial temperature value of the battery as an ambient temperature.

The average lithium concentration of the electrode active material is represented as:

c_{s,av}^{±}(0)=ƒ_{init,c}(cₘᵢₙ^{±},cₘₐₓ^{±},SOC₀)

The surface lithium concentration of the electrode active material is represented as:

c_{s,surf}^{±}(0)=c_{s,av}^{±}(0)

The lithium concentration of the electrode electrolyte is represented as:

cₑ(0)=ƒ_{init,e}(cₑ₀)

The initial temperature value of the battery is represented as:

T_{b}(0)=T_{amb}

where c_{s,av}^{±}(0) represents an initial value of the average lithium concentration of positive and negative electrode active materials, ƒ_{init,c} represents a set function of the initial value of the average lithium concentration of the electrode active materials, cₘᵢₙ^{±} represents a theoretical minimum value of the average lithium concentration of the positive and negative electrode active materials of the battery, cₘₐₓ^{±} represents a theoretical maximum value of the average lithium concentration of the positive and negative electrode active materials of the battery, SOC₀ represents the initial state of charge, c_{s,surf}^{±}(0) represents an initial value of the surface lithium concentration of the positive and negative electrode active materials, cₑ(0) represents an initial value of the lithium concentration of the electrode electrolyte, ƒ_{init,e} represents a set function of the initial value of the lithium concentration of the electrode electrolyte, cₑ₀ represents a material parameter of the lithium concentration of the electrode electrolyte, T_{b}(0) represents the initial temperature value of the battery, and T_{amb} represents the ambient temperature of the battery.

Further, in embodiments of the present disclosure, obtaining the port voltage of the battery at each moment within the preset time period based on the initial state information about the battery and the electrochemical model simulation of the lithium-ion battery includes:
obtaining an ambient temperature sequence of the battery with the constant amplitude, where the ambient temperature sequence of the battery includes an ambient temperature of the battery at each moment within the preset time period, where the ambient temperature sequence may be represented as:

$${T}_{amb} = {\underbrace{[ \begin{matrix}{T}_{amb} & … & {T}_{amb}\end{matrix} ]}}_{1 \times N}$$

where T_{amb} represents the ambient temperature of the battery, N represents the total number of moments in the preset time period, the period of the action of the ambient temperature of the battery at each moment is represented as tₖ≤t<tₖ₊₁, and the sampling interval is represented as Δt=tₖ₊₁−tₖ;
at a starting moment of the preset time period, updating a parameter vector at a current moment based on the lithium concentration of the electrode electrolyte, the average lithium concentration of the electrode active material and the battery temperature at a previous moment:

$$θ ( k + 1 ) = {f}_{θ} ( {c}_{e} ( k ) , {c}_{s , av} ( k ) , {T}_{b} ( k ) )$$

where θ(k+1) represents the parameter vector at the current moment, ƒ_{θ} represents a parameter update function, cₑ(k) represents the lithium concentration of the electrode electrolyte at the previous moment, c_{s,av}(k) represents the average lithium concentration of the electrode active material at the previous moment, and T_{b}(k) represents the battery temperature at the previous moment;
updating a reaction current intensity at the current moment based on the lithium concentration of the electrode electrolyte, the lithium concentration on the surface of the electrode active material, the battery temperature, and port current at the previous moment and the parameter vector at the current moment:

$${j}_{n} ( k + 1 ) = {f}_{j} ( {c}_{e} ( k ) , {c}_{s , surf} ( k ) , {T}_{b} ( k ) , I ( k ) , θ ( k + 1 ) )$$

where jₙ(k+1) represents the reaction current intensity at the current moment, ƒⱼ represents a reaction current update function, cₑ(k) represents the lithium concentration of the electrode electrolyte at the previous moment, c_{s,surf}(k) represents the lithium concentration on the surface of the electrode active material at the previous moment, T_{b}(k) represents the battery temperature at the previous moment, I(k) represents the port current at the previous moment, and θ(k+1) represents the parameter vector at the current moment;
updating a potential difference on a surface of an electrode at the current moment based on the reaction current intensity and the parameter vector at the current moment:

$${ϕ}_{se} ( k + 1 ) = {f}_{ϕ} ( {j}_{n} ( k + 1 ) , θ ( k + 1 ) )$$

where ϕₛₑ(k+1) represents the potential difference on the solid-solution surface of the electrode at the current moment, ƒ_{ϕ} represents an update function of the potential difference on the solid-solution surface of the electrode, jₙ(k+1) represents the reaction current intensity at the current moment, and θ(k+1) represents the parameter vector at the current moment;
updating the lithium concentration of the electrode active material at the current moment based on the average lithium concentration of the electrode active material and the lithium concentration on the surface of the electrode active material at the previous moment, the reaction current intensity at the current moment, the parameter vector at the current moment and a sampling interval:

$${c}_{s , av} ( k + 1 ) = {f}_{av} ( {c}_{s , av} ( k ) , {c}_{s , surf} ( k ) , {j}_{n} ( k + 1 ) , θ ( k + 1 ) , Δ t )$$

$${c}_{s , surf} ( k + 1 ) = {f}_{surf} ( {c}_{s , av} ( k ) , {c}_{s , surf} ( k ) , {j}_{n} ( k + 1 ) , θ ( k + 1 ) , Δ t )$$

where c_{s,av}(k+1) represents the average lithium concentration of the electrode active material at the current moment, ƒₐᵥ represents an update function of the average lithium concentration of the electrode active material, c_{s,av}(k) represents the average lithium concentration of the electrode active material at the previous moment, c_{s,surf}(k) represents the lithium concentration on the surface of the electrode active material at the previous moment, jₙ(k+1) represents the reaction current intensity at the current moment, θ(k+1) represents the parameter vector at the current moment, Δt represents the sampling interval, c_{s,surf}(k+1) represents the lithium concentration on the surface of the electrode active material at the current moment, ƒ_{surf} represents an update function of the lithium concentration on the surface of the electrode active material, c_{s,av}(k) represents the average lithium concentration of the electrode active material at the previous moment, c_{s,surf}(k) represents the lithium concentration on the surface of the electrode active material at the previous moment, jₙ(k+1) represents the reaction current intensity at the current moment, θ(k+1) represents the parameter vector at the current moment, and Δt represents the sampling interval;
updating the lithium concentration of the electrode electrolyte at the current moment based on the lithium concentration of the electrode electrolyte and the port current at the previous moment, the parameter vector at the current moment and the sampling interval:

$${c}_{e} ( k + 1 ) = {f}_{e} ( {c}_{e} ( k ) , I ( k ) , θ ( k + 1 ) , Δ t )$$

where cₑ(k+1) represents the lithium concentration of the electrode electrolyte at the current moment, ƒₑ represents an update function of the lithium concentration of the electrode electrolyte, cₑ(k) represents the lithium concentration of the electrode electrolyte at the previous moment, I(k) represents the port current at the previous moment, θ(k+1) represents the parameter vector at the current moment, and Δt represents the sampling interval;
obtaining a port voltage V of the battery and a potential difference U in the battery at the current moment based on the lithium concentration of the electrode electrolyte, the lithium concentration on the surface of the electrode active material, the reaction current intensity and the parameter vector at the current moment, and the battery temperature and the port current at the previous moment:

$$V ( k + 1 ) = {f}_{V} ( {c}_{e} ( k + 1 ) , {c}_{s , surf} ( k + 1 ) , {j}_{n} ( k + 1 ) , {T}_{b} ( k ) , I ( k ) , θ ( k + 1 ) )$$

$$U ( k + 1 ) = {f}_{U} ( {c}_{e} ( k + 1 ) , {c}_{s , surf} ( k + 1 ) , {j}_{n} ( k + 1 ) , {T}_{b} ( k ) , I ( k ) , θ ( k + 1 ) )$$

where V(k+1) represents the port voltage of the battery at the current moment, ƒ_{V} represents an update function of the port voltage of the battery, cₑ(k+1) represents the lithium concentration of the electrode electrolyte at the current moment, c_{s,surf}(k+1) represents the lithium concentration on the surface of the electrode active material at the current moment, jₙ(k+1) represents the reaction current intensity at the current moment, T_{b}(k) represents the battery temperature at the previous moment, I(k) represents the port current at the previous moment, θ(k+1) represents the parameter vector at the current moment, U(k+1) represents the potential difference in the battery at the current moment, ƒ_{U} represents an update function of the potential difference in the battery, cₑ(k+1) represents the lithium concentration of the electrode electrolyte at the current moment, c_{s,surf}(k+1) represents the lithium concentration on the surface of the electrode active material at the current moment, jₙ(k+1) represents the reaction current intensity at the current moment, T_{b}(k) represents the battery temperature at the previous moment, I(k) represents the port current at the previous moment, and θ(k+1) represents the parameter vector at the current moment;
obtaining the battery temperature at the current moment based on the port voltage of the battery, the potential difference in the battery, the reaction current intensity and the parameter vector at the current moment, and the battery temperature, ambient temperature, the port current and the sampling interval at the previous moment:

$${T}_{b} ( k + 1 ) = {f}_{T} ( V ( k + 1 ) , U ( k + 1 ) , {j}_{n} ( k + 1 ) , {T}_{b} ( k ) , {T}_{amb} ( k ) , I ( k ) , θ ( k + 1 ) , Δ t )$$

where T_{b}(k+1) represents the battery temperature at the current moment, ƒ_{T} represents an update function of the battery temperature, V(k+1) represents the port voltage of the battery at the current moment, U(k+1) represents the potential difference in the battery at the current moment, jₙ(k+1) represents the reaction current intensity at the current moment, T_{b}(k) represents the battery temperature at the previous moment, T_{amb}(k) represents the ambient temperature at the previous moment, I(k) represents the port current at the previous moment, θ(k+1) represents the parameter vector at the current moment, and Δt represents the sampling interval; and
repeating the above-mentioned simulation, iteration and update steps, and cyclically updating states at the current moment from states at the previous moment: the parameter vector, the reaction current intensity, the potential difference on the solid-solution surface of the electrode, the lithium concentration of the electrode active material, and the lithium concentration of the electrode electrolyte, and outputting the port voltage of the battery and the battery temperature based on a state update result until the preset time period ends to obtain the port voltage of the battery at each moment within the preset time period,
where the port voltage of the battery at each moment within the preset time period may be represented as

V=[V₁V₂ . . . Vₖ . . . V_{N}]
where V represents the port voltage of the battery at each moment within the preset time period, Vₖ represents a port voltage of the battery at a k^{th} moment, and V_{N} represents a port voltage of the battery at an N^{th} moment, and
the above-mentioned simulation, iteration and update may be represented as:

V=ƒ_{bat}(SOC₀,T_{amb},I)
where V represents the port voltage of the battery at each moment within the preset time period, ƒ_{bat} represents a state updating function set, SOC₀ represents the initial state of charge, T_{amb} represents the ambient temperature of the battery, and I represents the amplitude of the current sequence.

In the present disclosure, parameters such as the reaction current intensity, the potential difference on the solid-solution surface of the electrode, the lithium concentration of the electrode electrolyte, the lithium concentration on the surface of the electrode active material, and the average lithium concentration of the electrode active material are all vectors, and there are 8 sampling points in space at each moment, and specific examples are as follows:

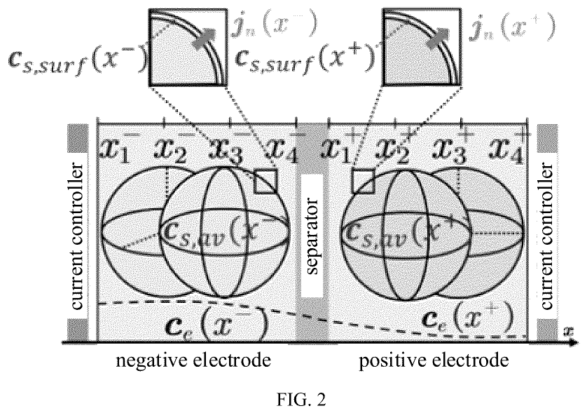

The reaction current intensity, the potential difference on the solid-solution surface of the electrode, the lithium concentration of the electrode electrolyte, the lithium concentration on the surface of the electrode active material, and the average lithium concentration of the electrode active material, by taking 4 sampling points at the positive and negative electrodes of the battery in the direction of increase in electrode thickness direction respectively, as shown in FIG. 2, are represented as:

$${j}_{n} ( k ) = [ {j}_{n} ( {x}_{1}^{\pm } , k ) , … , {j}_{n} ( {x}_{4}^{\pm } , k ) ]$$

$${ϕ}_{se} ( k ) = [ {ϕ}_{se} ( {x}_{1}^{\pm } , k ) , … , {ϕ}_{se} ( {x}_{4}^{\pm } , k ) ]$$

$${c}_{e} ( k ) = [ {c}_{e} ( {x}_{1}^{\pm } , k ) , … , {c}_{e} ( {x}_{4}^{\pm } , k ) ]$$

$${c}_{s , av} ( k ) = [ {c}_{s , av} ( {x}_{1}^{\pm } , k ) , … , {c}_{s , av} ( {x}_{4}^{\pm } , k ) ]$$

$${c}_{s , surf} ( k ) = [ {c}_{s , surf} ( {x}_{1}^{\pm } , k ) , … , {c}_{s , surf} ( {x}_{4}^{\pm } , k ) ]$$

where jₙ(k) represents the reaction current intensity at the current moment, jₙ(x₁^{±}, k) represents the reaction current intensity at sampling point 1 at the current moment, jₙ(x₄^{±}, k) represents the reaction current intensity at sampling point 4 at the current moment, ϕₛₑ(k) represents the potential difference on the solid-solution surface of the electrode at the current moment, ϕₛₑ(x₁^{±}, k) represents the potential difference on the solid-solution surface of the electrode at the sampling point 1 at the current moment, ϕₛₑ(x₄^{±}, k) represents the potential difference on the solid-solution surface of the electrode at the sampling point 4 at the current moment, cₑ(k) represents the lithium concentration of the electrode electrolyte at the current moment, cₑ(x₁^{±}, k) represents the lithium concentration of the electrode electrolyte at the sampling point 1 at the current moment, cₑ(x₄^{±}, k) represents the lithium concentration of the electrode electrolyte at the sampling point 4 at the current moment, c_{s,av}(k) represents the average lithium concentration of the electrode active material at the current moment, c_{s,av}(x₁^{±}, k) represents the average lithium concentration of the electrode active material at the sampling point 1 at the current moment, c_{s,av}(x₄^{±}, k) represents the average lithium concentration of the electrode active material at the sampling point 4 at the current moment, c_{s,surf}(k) represents the lithium concentration on the surface of the electrode active material at the current moment, c_{s,surf}(x₁^{±}, k) represents the lithium concentration on the surface of the electrode active material at the sampling point 1 at the current moment, and c_{s,surf}(x₄^{±}, k) represents the lithium concentration on the surface of the electrode active material at the sampling point 4 at the current moment.

Further, in embodiments of the present disclosure, calculating the port power corresponding to the initial state of charge based on the port voltage of the battery and the amplitude of the current sequence includes:
obtaining an average port voltage within the preset time period based on the port voltage of the battery, and calculating the port power corresponding to the initial state of charge and the amplitude of the current sequence based on the average port voltage and the amplitude of the current sequence;
where the average port voltage is represented as:

$$\overline{V} = \frac{{V}_{1} + {V}_{2} + … + {V}_{k} + … + {V}_{N}}{N}$$

where V̅ represents the average port voltage, Vₖ represents the port voltage of the battery at the k^{th} moment, V_{N} represents the port voltage of the battery at the N^{th} moment, and N represents the total number of moments in the preset time period; and
the port power is represented as:

$$P ( {SOC}_{0} , {T}_{amb} ) = I \times \overline{V}$$

where P represents the port power, SOC₀ represents the initial state of charge, T_{amb} represents a set ambient temperature of the battery, I represents the amplitude of the current sequence, and V̅ represents the average port voltage.

Further, in embodiments of the present disclosure, adjusting the initial state of charge and the amplitude of the current sequence, repeating the steps S1-S3 to respectively obtain port powers corresponding to the plurality of different initial states of charge and the plurality of different amplitudes of the current sequence, and obtaining the current amplitude-state of charge-port power curved surface based on the port powers comprise:
adjusting the initial state of charge and the amplitude of the current sequence of the battery, repeating the steps S1-S3 to respectively obtain port powers corresponding to the plurality of different initial states of charge and the plurality of different amplitudes of the current sequence, and smoothly connecting the port powers to obtain the current amplitude-state of charge-port power curved surface under a condition of maintaining the ambient temperature of the battery constant;
where the current amplitude-state of charge-port power curved surface is represented as:

$${f}_{P} ( {SOC}_{0} , P , I ) {\vert}_{T = {T}_{amb}} = 0$$

where ƒ_{P} represents a function of the current amplitude-state of charge-port power curved surface in a case of T=T_{amb}, SOC₀ represents the initial state of charge, P represents the port power, I represents the amplitude of the current sequence, T_{amb} represents the set ambient temperature of the battery, and T represents an actual ambient temperature of the battery.

Further, in embodiments of the present disclosure, fitting the current amplitude-state of charge-port power curved surface to the plane equation is represented as:

$${f}_{P} ( {SOC}_{0} , P , I ) {\vert}_{T = {T}_{amb}} \approx A \times {SOC}_{0} + B \times P + C \times I + D = 0$$

where ƒ_{P} represents the function of the current amplitude-state of charge-port power curved surface in the case of T=T_{amb}, SOC₀ represents the initial state of charge, P represents the port power, I represents the amplitude of the current sequence, T_{amb} represents the set ambient temperature of the battery, T represents the actual ambient temperature of the battery, A, B and C are first-order coefficients of plane fitting, and D is a constant coefficient of the plane fitting.

Further, in embodiments of the present disclosure, obtaining the current amplitude corresponding to the port power and the state of charge by using the plane equation based on the port power and the state of charge, and updating the state of charge based on the current amplitude and the preset time period comprise:
obtaining the current amplitude by using the plane equation based on the known port power and state of charge under the condition of maintaining the ambient temperature of the battery constant;

where the current amplitude is represented as:

$$I = {f}_{I} ( {SOC}_{0} , P ) {\vert}_{T = {T}_{amb}} \approx {a}_{0} ( {T}_{amb} ) + {a}_{1} ( {T}_{amb} ) \times {SOC}_{0} + {a}_{2} ( {T}_{amb} ) \times P$$

where I represents the current amplitude, ƒ_{I} represents a current amplitude function, SOC₀ represents the initial state of charge, T_{amb} represents the set ambient temperature of the battery, T represents the actual ambient temperature of the battery, a₀ represents a constant coefficient of the current amplitude function, a₁ represents a first-order coefficient corresponding to SOC₀ in the current magnitude function, a₂ represents a first-order coefficient corresponding to P in the current magnitude function, and P represents the port power;
a₀, a₁, a₂ may be derived from the coefficients A, B, C, D of the plane fitting:

$${a}_{0} = - \frac{D}{C}$$

$${a}_{1} = - \frac{A}{C}$$

$${a}_{2} = - \frac{B}{C}$$

where A, B and C are first-order coefficients of plane fitting, D is the constant coefficient of the plane fitting, do represents the constant coefficient of the current amplitude function, a₁ represents the first-order coefficient corresponding to SOC₀ in the current magnitude function, and a₂ represents the first-order coefficient corresponding to P in the current magnitude function; and
updating the state of charge based on the current amplitude and the preset time period, which is represented as:

$$Δ SOC = \frac{- I \times Δ T}{{C}_{0}}$$

$$Δ T = N Δ t$$

$${SOC}_{T + 1} = {SOC}_{T} + Δ SOC$$

where ΔSOC represents a change in the state of charge in adjacent time periods, I represents the current amplitude, C₀ represents a total capacity of the battery in ampere-hour, ΔT represents a length of the preset time period, N represents the total number of moments in the preset time period, Δt represents the sampling interval, SOC_{T+1} represents the state of charge of the battery at start of a next time period, and SOC represents the state of charge of the battery at start of a present time period.

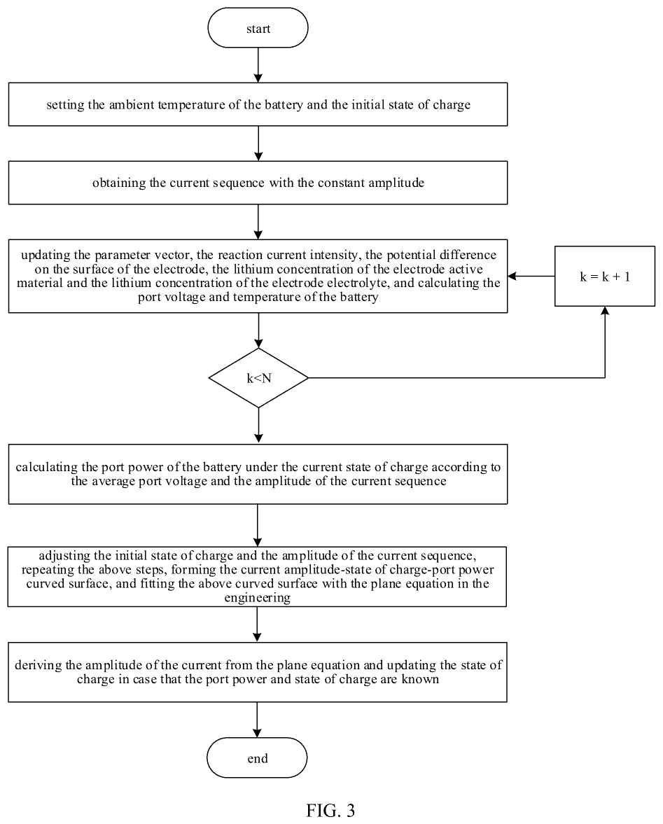

FIG. 3 is another flow chart illustrating a method for updating a state of charge based on power characteristics of an electrochemical model of a lithium-ion battery in embodiments of the present disclosure.

As shown in FIG. 3, the ambient temperature of the battery and the initial state of charge are preset to obtain the current sequence with the constant amplitude. The electrochemical model simulation is performed, the parameter vector, the reaction current intensity, the potential difference on the solid-solution surface of the electrode, the lithium concentration of the electrode active material and the lithium concentration of the electrode electrolyte are updated, and the port voltage and temperature of the battery are calculated according to preset parameters and the current sequence with the constant amplitude. Port average power under the current state of charge and the current amplitude is calculated according to the calculated average value of the port voltage and the amplitude of the current sequence. The initial state of charge and the amplitude of the current sequence are adjusted, and the above-mentioned steps are repeated to form the current amplitude-state of charge-port power curved surface, and the above-mentioned curved surface is fitted with the plane equation in the engineering. In case that the state of charge is updated, the amplitude of the current sequence may be derived from the above-mentioned plane equation from the known port power and initial state of charge of the battery, and the updating of the state of charge may be performed using the obtained amplitude.

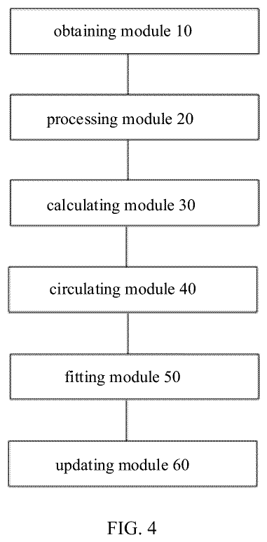

FIG. 4 is a schematic block diagram illustrating a device for updating a state of charge based on power characteristics of an electrochemical model of a lithium-ion battery provided in Embodiment 2 of the present disclosure.

As shown in FIG. 4, the device for updating the state of charge based on the power characteristics of the electrochemical model of the lithium-ion battery includes:
an obtaining module 10 configured to obtain the initial state of charge of a battery and the current sequence with the constant amplitude;
a processing module 20 configured to obtain the initial state information about the battery, and obtain the port voltage of the battery at each moment within the preset time period based on the initial state information about the battery and the electrochemical model simulation of the lithium-ion battery;
a calculating module 30 configured to calculate the port power corresponding to the initial state of charge based on the port voltage of the battery and the amplitude of the current sequence;
a circulating module 40 configured to adjust the initial state of charge and the amplitude of the current sequence, repeat invoking the obtaining module, the processing module and the calculating module to respectively obtain port powers corresponding to the multiple different initial states of charge and the multiple different amplitudes of the current sequence, and obtain the current amplitude-state of charge-port power curved surface based on the port powers;
a fitting module 50 configured to fit the current amplitude-state of charge-port power curved surface to a plane equation; and
an updating module 60 configured to obtain the current amplitude corresponding to the port power and the state of charge by using the plane equation based on the port power and the state of charge, and update the state of charge based on the current amplitude and the preset time period.

The device for updating the state of charge based on the power characteristics of the electrochemical model of the lithium-ion battery in embodiments of the present disclosure includes the obtaining module configured to obtain the initial state of charge of the battery and the current sequence with the constant amplitude; a processing module configured to obtain the initial state information about the battery, and obtain the port voltage of the battery at each moment within the preset time period based on the initial state information about the battery and the electrochemical model simulation of the lithium-ion battery; a calculating module configured to calculate the port power corresponding to the initial state of charge based on the port voltage of the battery and the amplitude of the current sequence; a circulating module configured to adjust the initial state of charge and the amplitude of the current sequence, repeat invoking the obtaining module, the processing module and the calculating module to respectively obtain the port powers corresponding to the multiple different initial states of charge and the multiple different amplitudes of the current sequence, and obtain the current amplitude-state of charge-port power curved surface based on the port powers; a fitting module configured to fit the current amplitude-state of charge-port power curved surface to the plane equation; and an updating module configured to obtain the current amplitude corresponding to the port power and the state of charge by using the plane equation based on the port power and the state of charge, and update the state of charge based on the current amplitude and the preset time period. In this way, the technical problem of mutual exclusion of the precision and efficiency for updating the state of charge of the lithium-ion battery in the existing method is solved. By utilizing the high simulation precision of the external characteristics of the electrochemical model of the lithium-ion battery and taking into account the influence of the time-varying port voltage of the battery on updating of the state of charge without increasing the computational complexity, the updating of the state of charge of the battery is achieved without performing the continuous time sequence simulation in case that the power and the state of charge of the battery are known, which improves the computational efficiency of the electrochemical model in the power application scenarios and expands the application scenarios of the electrochemical model in the engineering.

In order to implement the above embodiments, the present disclosure further proposes an electronic device, including: a memory, a processor, and a computer program stored in the memory and executable on the processor, in which the computer program, when executed by the processor, implements the above-mentioned method for updating the state of charge based on the power characteristics of the electrochemical model of the lithium-ion battery.

In order to implement the above embodiments, the present disclosure further proposes a non-transitory computer readable storage medium having stored thereon a computer program, in which the computer program, when executed by a processor, implements the above-mentioned method for updating the state of charge based on the power characteristics of the electrochemical model of the lithium-ion battery.

In order to implement the above embodiments, the present disclosure further proposes a computer program product, comprising a computer program, in which the computer program, when executed by a processor, implements the above-mentioned method for updating the state of charge based on the power characteristics of the electrochemical model of the lithium-ion battery.

With the method and the device for updating the state of charge based on the power characteristics of the electrochemical model of the lithium-ion battery and the non-transitory computer readable storage medium according to embodiments of the present disclosure, the technical problem of mutual exclusion of the precision and efficiency for updating the state of charge of the lithium-ion battery in the existing method is solved. By utilizing high simulation precision of the external characteristics of the electrochemical model of the lithium-ion battery and taking into account the influence of the time-varying port voltage of the battery on updating of the state of charge without increasing computational complexity, the updating of the state of charge of the battery is achieved without performing continuous time sequence simulation in case that the power and the state of charge of the battery are known, which improves computational efficiency of the electrochemical model in power application scenarios and expands the application scenarios of the electrochemical model in engineering.

Reference throughout this specification to terms such as “an embodiment,” “some embodiments,” “an example,” “a specific example,” or “some examples,” means that a particular feature, structure, material, or characteristic described in connection with the embodiment or example is included in at least one embodiment or example of the present disclosure. Schematic expressions of the above terms throughout this specification are not necessarily referring to the same embodiment or example. Furthermore, the particular features, structures, materials, or characteristics described may be combined in any suitable manner in one or more embodiments or examples. In addition, different embodiments or examples and features of different embodiments or examples described in the specification may be combined by those skilled in the art without mutual contradiction.

In addition, terms such as “first” and “second” are used herein for purposes of description and are not intended to indicate or imply relative importance or implicitly specify the number of technical features indicated. Thus, the feature defined with “first” and “second” may explicitly or implicitly comprise one or more of this feature. In the description of the present disclosure, “a plurality of” means at least two, for example, two or three, unless specified otherwise.

Any process or method described in a flow chart or described herein in other manners may be understood to include one or more modules, segments or portions of codes of executable instructions for achieving specific logical functions or steps in the process, and the scope of a preferred embodiment of the present disclosure includes other implementations, in which the functions may be performed out of the order shown or discussed, including in a substantially simultaneous manner or in a reverse order depending on the functionality involved, which should be understood by those skilled in the art.

The logic and/or step shown in the flow chart or described herein in other manners, for example, a particular sequence table of executable instructions for realizing the logical function, may be specifically achieved in any computer readable medium to be used by the instruction execution system, device or equipment (such as the system based on computers, the system comprising processors or other systems capable of obtaining the instruction from the instruction execution system, device or equipment and executing the instruction), or to be used in combination with the instruction execution system, device or equipment. As to the specification, “the computer readable medium” may be any device adaptive for including, storing, communicating, propagating or transferring programs to be used by or in combination with the instruction execution system, device or equipment. More specific examples (non-exhaustive list) of the computer readable medium comprise but are not limited to: an electronic connection (an electronic device) with one or more wires, a portable computer enclosure (a magnetic device), a random access memory (RAM), a read only memory (ROM), an erasable programmable read-only memory (EPROM or a flash memory), an optical fiber device and a portable compact disk read-only memory (CDROM). In addition, the computer readable medium may even be a paper or other appropriate medium capable of printing programs thereon, because, for example, the paper or other appropriate medium may be optically scanned and then edited, decrypted or processed with other appropriate methods when necessary to obtain the programs in an electric manner, and then the programs may be stored in the computer memories.

It should be understood that each part of the present disclosure may be realized by the hardware, software, firmware or their combination. In the above embodiments, a plurality of steps or methods may be realized by the software or firmware stored in the memory and executed by the appropriate instruction execution system. For example, if they are realized by the hardware, likewise in another embodiment, the steps or methods may be realized by any one or a combination of the following techniques known in the art: a discrete logic circuit having a logic gate circuit for realizing a logic function of a data signal, an application-specific integrated circuit having an appropriate combination logic gate circuit, a programmable gate array (PGA), a field programmable gate array (FPGA), etc.

It would be understood by those skilled in the art that all or some of the steps carried by the method in the above-described embodiments may be completed by relevant hardware instructed by a program. The program may be stored in a computer readable storage medium. When the program is executed, one or a combination of the steps of the method in the above-described embodiments may be completed.

In addition, individual functional units in the embodiments of the present disclosure may be integrated in one processing module or may be separately physically present, or two or more units may be integrated in one module. The integrated module as described above may be achieved in the form of hardware, or may be achieved in the form of a software functional module. If the integrated module is achieved in the form of a software functional module and sold or used as a separate product, the integrated module may also be stored in a computer readable storage medium.

The storage medium mentioned above may be read-only memories, magnetic disks CD, or the like.

Although embodiments of the present disclosure have been shown and described, it would be appreciated by those skilled in the art that the above embodiments are exemplary and cannot be construed to limit the present disclosure, and changes, modifications, alternatives, and variations can be made in the above embodiments without departing from the scope of the present disclosure.

What is claimed is:

1. A method for updating a state of charge based on power characteristics of an electrochemical model of a lithium-ion battery, comprising:
S1: obtaining an initial state of charge of a battery and a current sequence with a constant amplitude;
S2: obtaining initial state information about the battery, and obtaining a port voltage of the battery at each moment within a preset time period based on the initial state information about the battery and an electrochemical model simulation of the lithium-ion battery;
S3: calculating port power corresponding to the initial state of charge based on the port voltage of the battery and the amplitude of the current sequence;
S4: adjusting the initial state of charge and the amplitude of the current sequence, repeating steps S1-S3 to respectively obtain port powers corresponding to a plurality of different initial states of charge and a plurality of different amplitudes of the current sequence, and obtaining a current amplitude-state of charge-port power curved surface based on the port powers;
S5: fitting the current amplitude-state of charge-port power curved surface to a plane equation; and
S6: obtaining a current amplitude corresponding to the port power and the state of charge by using the plane equation based on the port power and the state of charge, and updating the state of charge based on the current amplitude and the preset time period.

2. The method of claim 1, wherein the initial state information about the battery comprises: a lithium concentration on a surface of an electrode active material, an average lithium concentration of the electrode active material, a lithium concentration of an electrode electrolyte, and an initial temperature of the battery.

3. The method of claim 2, wherein obtaining the port voltage of the battery at each moment within the preset time period based on the initial state information about the battery and the electrochemical model simulation of the lithium-ion battery comprises:
obtaining an ambient temperature sequence of the battery with a constant amplitude, wherein the ambient temperature sequence of the battery comprises an ambient temperature of the battery at each moment within the preset time period,
at a starting moment of the preset time period, updating a parameter vector at a current moment based on the lithium concentration of the electrode electrolyte, the average lithium concentration of the electrode active material and the battery temperature at a previous moment:
$$θ ( k + 1 ) = {f}_{θ} ( {c}_{e} ( k ) , {c}_{s , av} ( k ) , {T}_{b} ( k ) )$$
where θ(k+1) represents the parameter vector at the current moment, ƒ_{θ} represents a parameter update function, cₑ(k) represents the lithium concentration of the electrode electrolyte at the previous moment, c_{s,av}(k) represents the average lithium concentration of the electrode active material at the previous moment, and T_{b}(k) represents the battery temperature at the previous moment;
updating a reaction current intensity at the current moment based on the lithium concentration of the electrode electrolyte, the lithium concentration on the surface of the electrode active material, the battery temperature, and port current at the previous moment and the parameter vector at the current moment:
$${j}_{n} ( k + 1 ) = {f}_{j} ( {c}_{e} ( k ) , {c}_{s , surf} ( k ) , {T}_{b} ( k ) , I ( k ) , θ ( k + 1 ) )$$
where jₙ(k+1) represents the reaction current intensity at the current moment, ƒⱼ represents a reaction current update function, c_{s,surf}(k) represents the lithium concentration on the surface of the electrode active material at the previous moment, and I(k) represents the port current at the previous moment;
updating a potential difference on a solid-solution surface of an electrode at the current moment based on the reaction current intensity and the parameter vector at the current moment:
$${ϕ}_{se} ( k + 1 ) = {f}_{ϕ} ( {j}_{n} ( k + 1 ) , θ ( k + 1 ) )$$
where ϕₛₑ(k+1) represents the potential difference on the solid-solution surface of the electrode at the current moment, and ƒ_{ϕ} represents an update function of the potential difference on the solid-solution surface of the electrode;
updating the lithium concentration of the electrode active material at the current moment based on the average lithium concentration of the electrode active material and the lithium concentration on the surface of the electrode active material at the previous moment, the reaction current intensity at the current moment, the parameter vector at the current moment and a sampling interval:
$${c}_{s , av} ( k + 1 ) = {f}_{av} ( {c}_{s , av} ( k ) , {c}_{s , surf} ( k ) , {j}_{n} ( k + 1 ) , θ ( k + 1 ) , Δ t )$$
$${c}_{s , surf} ( k + 1 ) = {f}_{surf} ( {c}_{s , av} ( k ) , {c}_{s , surf} ( k ) , {j}_{n} ( k + 1 ) , θ ( k + 1 ) , Δ t )$$
where c_{s,av}(k+1) represents the average lithium concentration of the electrode active material at the current moment, ƒₐᵥ represents an update function of the average lithium concentration of the electrode active material, Δt represents the sampling interval, c_{s,surf}(k+1) represents the lithium concentration on the surface of the electrode active material at the current moment, and ƒ_{surf} represents an update function of the lithium concentration on the surface of the electrode active material;
updating the lithium concentration of the electrode electrolyte at the current moment based on the lithium concentration of the electrode electrolyte and the port current at the previous moment, the parameter vector at the current moment and the sampling interval:
$${c}_{e} ( k + 1 ) = {f}_{e} ( {c}_{e} ( k ) , I ( k ) , θ ( k + 1 ) , Δ t )$$
where cₑ(k+1) represents the lithium concentration of the electrode electrolyte at the current moment, and ƒₑ represents an update function of the lithium concentration of the electrode electrolyte;
obtaining a port voltage V of the battery and a potential difference U in the battery at the current moment based on the lithium concentration of the electrode electrolyte, the lithium concentration on the surface of the electrode active material, the reaction current intensity and the parameter vector at the current moment, and the battery temperature and the port current at the previous moment:
$$V ( k + 1 ) = {f}_{V} ( {c}_{e} ( k + 1 ) , {c}_{s , surf} ( k + 1 ) , {j}_{n} ( k + 1 ) , {T}_{b} ( k ) , I ( k ) , θ ( k + 1 ) )$$
$$U ( k + 1 ) = {f}_{U} ( {c}_{e} ( k + 1 ) , {c}_{s , surf} ( k + 1 ) , {j}_{n} ( k + 1 ) , {T}_{b} ( k ) , I ( k ) , θ ( k + 1 ) )$$
where V(k+1) represents the port voltage of the battery at the current moment, ƒ_{V} represents an update function of the port voltage of the battery, U(k+1) represents the potential difference in the battery at the current moment, and ƒ_{U} represents an update function of the potential difference in the battery;
obtaining the battery temperature at the current moment based on the port voltage of the battery, the potential difference in the battery, the reaction current intensity and the parameter vector at the current moment, and the battery temperature, ambient temperature, the port current and the sampling interval at the previous moment:
$${T}_{b} ( k + 1 ) = {f}_{T} ( V ( k + 1 ) , U ( k + 1 ) , {j}_{n} ( k + 1 ) , {T}_{b} ( k ) , {T}_{amb} ( k ) , I ( k ) , θ ( k + 1 ) , Δ t )$$
where T_{b}(k+1) represents the battery temperature at the current moment, ƒ_{T} represents an update function of the battery temperature, and T_{amb}(k) represents the ambient temperature at the previous moment; and
repeating the above-mentioned simulation, iteration and update steps, and cyclically updating states at the current moment from states at the previous moment: the parameter vector, the reaction current intensity, the potential difference on the solid-solution surface of the electrode, the lithium concentration of the electrode active material, and the lithium concentration of the electrode electrolyte, and outputting the port voltage of the battery and the battery temperature based on a state update result until the preset time period ends to obtain the port voltage of the battery at each moment within the preset time period,
wherein the port voltage of the battery at each moment within the preset time period may be represented as:
V=[V₁V₂ . . . Vₖ . . . V_{N}]
where V represents the port voltage of the battery at each moment within the preset time period, Vₖ represents a port voltage of the battery at a k^{th} moment, and N is a total number of moments within the preset time period, wherein N is an integer greater than or equal to 2.

4. The method of claim 3, wherein calculating the port power corresponding to the initial state of charge based on the port voltage of the battery and the amplitude of the current sequence comprises:
obtaining an average port voltage within the preset time period based on the port voltage of the battery, and calculating the port power corresponding to the initial state of charge and the amplitude of the current sequence based on the average port voltage and the amplitude of the current sequence;
wherein the average port voltage is represented as:
$$\overline{V} = \frac{{V}_{1} + {V}_{2} + … + {V}_{k} + … + {V}_{N}}{N}$$
where V̅ represents the average port voltage; and
the port power is represented as:
$$P ( {SOC}_{0} , {T}_{amb} ) = I \times \overline{V}$$
where P represents the port power, SOC₀ represents the initial state of charge, T_{amb} represents a set ambient temperature of the battery, and I represents the amplitude of the current sequence.

5. The method of claim 4, wherein adjusting the initial state of charge and the amplitude of the current sequence, repeating the steps S1-S3 to respectively obtain port powers corresponding to the plurality of different initial states of charge and the plurality of different amplitudes of the current sequence, and obtaining the current amplitude-state of charge-port power curved surface based on the port powers comprise:
adjusting the initial state of charge and the amplitude of the current sequence of the battery, repeating the steps S1-S3 to respectively obtain port powers corresponding to the plurality of different initial states of charge and the plurality of different amplitudes of the current sequence, and smoothly connecting the port powers to obtain the current amplitude-state of charge-port power curved surface under a condition of maintaining the ambient temperature of the battery constant;
wherein the current amplitude-state of charge-port power curved surface is represented as:
$${f}_{P} ( {SOC}_{0} , P , I ) {\vert}_{T = {T}_{amb}} = 0$$
where ƒ_{P} represents a function of the current amplitude-state of charge-port power curved surface in a case of T=T_{amb}, and T represents an actual ambient temperature of the battery.

6. The method of claim 5, wherein fitting the current amplitude-state of charge-port power curved surface to the plane equation is represented as:
$${f}_{P} ( {SOC}_{0} , P , I ) {\vert}_{T = {T}_{amb}} \approx A \times {SOC}_{0} + B \times P + C \times I + D = 0$$
where ƒ_{P} represents the function of the current amplitude-state of charge-port power curved surface in the case of T=T_{amb}, A, B and C are first-order coefficients of plane fitting, and D is a constant coefficient of the plane fitting.

7. The method of claim 6, wherein obtaining the current amplitude corresponding to the port power and the state of charge by using the plane equation based on the port power and the state of charge, and updating the state of charge based on the current amplitude and the preset time period comprise:
obtaining the current amplitude by using the plane equation based on the known port power and state of charge under the condition of maintaining the ambient temperature of the battery constant;
wherein the current amplitude is represented as:
$$I = {f}_{I} ( {SOC}_{0} , P ) {\vert}_{T = {T}_{amb}} \approx {a}_{0} ( {T}_{amb} ) + {a}_{1} ( {T}_{amb} ) \times {SOC}_{0} + {a}_{2} ( {T}_{amb} ) \times P$$
where I represents the current amplitude, ƒ_{I} represents a current amplitude function, a₀ represents a constant coefficient of the current amplitude function, a₁ represents a first-order coefficient corresponding to SOC₀ in the current magnitude function, a₂ represents a first-order coefficient corresponding to P in the current magnitude function;
a₀, a₁, a₂ are derived from the coefficients A, B, C, D of the plane fitting:
$${a}_{0} = - \frac{D}{C}$$
$${a}_{1} = - \frac{A}{C}$$
$${a}_{2} = - \frac{B}{C} ,$$
and
updating the state of charge based on the current amplitude and the preset time period, which is represented as:
$$Δ SOC = \frac{- I \times Δ T}{{C}_{0}}$$
$$Δ T = N Δ t$$
$${SOC}_{T + 1} = {SOC}_{T} + Δ SOC$$
where ΔSOC represents a change in the state of charge in adjacent time periods, C₀ represents a total capacity of the battery in ampere-hour, ΔT represents a length of the preset time period, SOC_{T+1} represents the state of charge of the battery at start of a next time period, and SOC_{T} represents the state of charge of the battery at start of a present time period.

8. An electronic device, comprising:
a memory; and
a processor storing a computer program, which, when executed by the processor, the processor is configured to:
obtain an initial state of charge of a battery and a current sequence with a constant amplitude;
obtain initial state information about the battery, and obtain a port voltage of the battery at each moment within a preset time period based on the initial state information about the battery and an electrochemical model simulation of a lithium-ion battery;
calculate port power corresponding to the initial state of charge based on the port voltage of the battery and the amplitude of the current sequence;
adjust the initial state of charge and the amplitude of the current sequence, repeat above steps to respectively obtain port powers corresponding to a plurality of different initial states of charge and a plurality of different amplitudes of the current sequence, and obtain a current amplitude-state of charge-port power curved surface based on the port powers;
fit the current amplitude-state of charge-port power curved surface to a plane equation; and
obtain a current amplitude corresponding to the port power and the state of charge by using the plane equation based on the port power and the state of charge, and update the state of charge based on the current amplitude and the preset time period.

9. The electronic device of claim 8, wherein the initial state information about the battery comprises: a lithium concentration on a surface of an electrode active material, an average lithium concentration of the electrode active material, a lithium concentration of an electrode electrolyte, and an initial temperature of the battery.

10. The electronic device of claim 9, wherein the processor is further configured to:
obtain an ambient temperature sequence of the battery with a constant amplitude, wherein the ambient temperature sequence of the battery comprises an ambient temperature of the battery at each moment within the preset time period,
at a starting moment of the preset time period, update a parameter vector at a current moment based on the lithium concentration of the electrode electrolyte, the average lithium concentration of the electrode active material and the battery temperature at a previous moment:
$$θ ( k + 1 ) = {f}_{θ} ( {c}_{e} ( k ) , {c}_{s , av} ( k ) , {T}_{b} ( k ) )$$
where θ(k+1) represents the parameter vector at the current moment, ƒₑ represents a parameter update function, cₑ(k) represents the lithium concentration of the electrode electrolyte at the previous moment, c_{s,av}(k) represents the average lithium concentration of the electrode active material at the previous moment, and T_{b}(k) represents the battery temperature at the previous moment;
update a reaction current intensity at the current moment based on the lithium concentration of the electrode electrolyte, the lithium concentration on the surface of the electrode active material, the battery temperature, and port current at the previous moment and the parameter vector at the current moment:
$${j}_{n} ( k + 1 ) = {f}_{j} ( {c}_{e} ( k ) , {c}_{s , surf} ( k ) , {T}_{b} ( k ) , I ( k ) , θ ( k + 1 ) )$$
where jₙ(k+1) represents the reaction current intensity at the current moment, ƒⱼ represents a reaction current update function, c_{s,surf}(k) represents the lithium concentration on the surface of the electrode active material at the previous moment, and I(k) represents the port current at the previous moment;
update a potential difference on a surface of an electrode at the current moment based on the reaction current intensity and the parameter vector at the current moment:
$${ϕ}_{se} ( k + 1 ) = {f}_{ϕ} ( {j}_{n} ( k + 1 ) , θ ( k + 1 ) )$$
where ϕₛₑ(k+1) represents the potential difference on the solid-solution surface of the electrode at the current moment, and ƒ_{ϕ} represents an update function of the potential difference on the solid-solution surface of the electrode;
update the lithium concentration of the electrode active material at the current moment based on the average lithium concentration of the electrode active material and the lithium concentration on the surface of the electrode active material at the previous moment, the reaction current intensity at the current moment, the parameter vector at the current moment and a sampling interval:
$${c}_{s , av} ( k + 1 ) = {f}_{av} ( {c}_{s , av} ( k ) , {c}_{s , surf} ( k ) , {j}_{n} ( k + 1 ) , θ ( k + 1 ) , Δ t )$$
$${c}_{s , surf} ( k + 1 ) = {f}_{surf} ( {c}_{s , av} ( k ) , {c}_{s , surf} ( k ) , {j}_{n} ( k + 1 ) , θ ( k + 1 ) , Δ t )$$
where c_{s,av}(k+1) represents the average lithium concentration of the electrode active material at the current moment, ƒₐᵥ represents an update function of the average lithium concentration of the electrode active material, Δt represents the sampling interval, c_{s,surf}(k+1) represents the lithium concentration on the surface of the electrode active material at the current moment, and ƒ_{surf} represents an update function of the lithium concentration on the surface of the electrode active material;
update the lithium concentration of the electrode electrolyte at the current moment based on the lithium concentration of the electrode electrolyte and the port current at the previous moment, the parameter vector at the current moment and the sampling interval:
$${c}_{e} ( k + 1 ) = {f}_{e} ( {c}_{e} ( k ) , I ( k ) , θ ( k + 1 ) , Δ t )$$
where cₑ(k+1) represents the lithium concentration of the electrode electrolyte at the current moment, and ƒₑ represents an update function of the lithium concentration of the electrode electrolyte;
obtain a port voltage V of the battery and a potential difference U in the battery at the current moment based on the lithium concentration of the electrode electrolyte, the lithium concentration on the surface of the electrode active material, the reaction current intensity and the parameter vector at the current moment, and the battery temperature and the port current at the previous moment:
$$V ( k + 1 ) = {f}_{V} ( {c}_{e} ( k + 1 ) , {c}_{s , surf} ( k + 1 ) , {j}_{n} ( k + 1 ) , {T}_{b} ( k ) , I ( k ) , θ ( k + 1 ) )$$
$$U ( k + 1 ) = {f}_{U} ( {c}_{e} ( k + 1 ) , {c}_{s , surf} ( k + 1 ) , {j}_{n} ( k + 1 ) , {T}_{b} ( k ) , I ( k ) , θ ( k + 1 ) )$$
where V(k+1) represents the port voltage of the battery at the current moment, ƒ_{V} represents an update function of the port voltage of the battery, U(k+1) represents the potential difference in the battery at the current moment, and ƒ_{U} represents an update function of the potential difference in the battery;
obtain the battery temperature at the current moment based on the port voltage of the battery, the potential difference in the battery, the reaction current intensity and the parameter vector at the current moment, and the battery temperature, ambient temperature, the port current and the sampling interval at the previous moment:
$${T}_{b} ( k + 1 ) = {f}_{T} ( V ( k + 1 ) , U ( k + 1 ) , {j}_{n} ( k + 1 ) , {T}_{b} ( k ) , {T}_{amb} ( k ) , I ( k ) , θ ( k + 1 ) , Δ t )$$
where T_{b}(k+1) represents the battery temperature at the current moment, ƒ_{T} represents an update function of the battery temperature, and T_{amb}(k) represents the ambient temperature at the previous moment; and
repeat the above-mentioned simulation, iteration and update steps, and cyclically updating states at the current moment from states at the previous moment: the parameter vector, the reaction current intensity, the potential difference on the solid-solution surface of the electrode, the lithium concentration of the electrode active material, and the lithium concentration of the electrode electrolyte, and output the port voltage of the battery and the battery temperature based on a state update result until the preset time period ends to obtain the port voltage of the battery at each moment within the preset time period,
wherein the port voltage of the battery at each moment within the preset time period may be represented as:
V=[V₁V₂ . . . Vₖ . . . V_{N}]
where V represents the port voltage of the battery at each moment within the preset time period, Vₖ represents a port voltage of the battery at a k^{th} moment, and N is a total number of moments within the preset time period, wherein N is an integer greater than or equal to 2.

11. The electronic device of claim 10, wherein the processor is further configured to:
obtain an average port voltage within the preset time period based on the port voltage of the battery, and calculate the port power corresponding to the initial state of charge and the amplitude of the current sequence based on the average port voltage and the amplitude of the current sequence;
wherein the average port voltage is represented as:
$$\overline{V} = \frac{{V}_{1} + {V}_{2} + … + {V}_{k} + … + {V}_{N}}{N}$$
where V̅ represents the average port voltage; and
the port power is represented as:
$$P ( {SOC}_{0} , {T}_{amb} ) = I \times \overline{V}$$
where P represents the port power, SOC₀ represents the initial state of charge, T_{amb} represents a set ambient temperature of the battery, and I represents the amplitude of the current sequence.

12. The electronic device of claim 11, wherein the processor is further configured to:
adjust the initial state of charge and the amplitude of the current sequence of the battery, repeat the above steps to respectively obtain port powers corresponding to the plurality of different initial states of charge and the plurality of different amplitudes of the current sequence, and smoothly connect the port powers to obtain the current amplitude-state of charge-port power curved surface under a condition of maintaining the ambient temperature of the battery constant;
wherein the current amplitude-state of charge-port power curved surface is represented as:
$${f}_{P} ( {SOC}_{0} , P , I ) {\vert}_{T = {T}_{amb}} = 0$$
where ƒ_{P} represents a function of the current amplitude-state of charge-port power curved surface in a case of T=T_{amb}, and T represents an actual ambient temperature of the battery.

13. The electronic device of claim 12, wherein fitting the current amplitude-state of charge-port power curved surface to the plane equation is represented as:
$${{f}_{P} ( {SOC}_{0} , P , I ) \vert}_{T = {T}_{amb}} \approx A \times {SOC}_{0} + B \times P + C \times I + D = 0$$
where ƒ_{P} represents the function of the current amplitude-state of charge-port power curved surface in the case of T=T_{amb}, A, B and C are first-order coefficients of plane fitting, and D is a constant coefficient of the plane fitting.

14. The electronic device of claim 13, wherein the processor is further configured to:
obtain the current amplitude by using the plane equation based on the known port power and state of charge under the condition of maintaining the ambient temperature of the battery constant;
wherein the current amplitude is represented as:
$${I = {f}_{I} ( {SOC}_{0} , P ) \vert}_{T = {T}_{amb}} \approx {a}_{0} ( {T}_{amb} ) + {a}_{1} ( {T}_{amb} ) \times {SOC}_{0} + {a}_{2} ( {T}_{amb} ) \times P$$
where I represents the current amplitude, ƒ_{I} represents a current amplitude function, a₀ represents a constant coefficient of the current amplitude function, a₁ represents a first-order coefficient corresponding to SOC₀ in the current magnitude function, a₂ represents a first-order coefficient corresponding to P in the current magnitude function;
a₀, a₁, a₂ are derived from the coefficients A, B, C, D of the plane fitting:
$${a}_{0} = - \frac{D}{C}$$
$${a}_{1} = - \frac{A}{C}$$
$${a}_{2} = - \frac{B}{C} ,$$
and
update the state of charge based on the current amplitude and the preset time period, which is represented as:
$$Δ SOC = \frac{- I \times Δ T}{{C}_{0}}$$
$$Δ T = N Δ t$$
$${SOC}_{T + 1} = {SOC}_{T} + Δ SOC$$
where ΔSOC represents a change in the state of charge in adjacent time periods, C₀ represents a total capacity of the battery in ampere-hour, ΔT represents a length of the preset time period, SOC_{T+1} represents the state of charge of the battery at start of a next time period, and SOC_{T} represents the state of charge of the battery at start of a present time period.

15. A non-transitory computer readable storage medium storing a computer program, which, when executed by a processor, the processor is configured to:
obtain an initial state of charge of a battery and a current sequence with a constant amplitude;
obtain initial state information about the battery, and obtain a port voltage of the battery at each moment within a preset time period based on the initial state information about the battery and an electrochemical model simulation of a lithium-ion battery;
calculate port power corresponding to the initial state of charge based on the port voltage of the battery and the amplitude of the current sequence;
adjust the initial state of charge and the amplitude of the current sequence, repeat above steps to respectively obtain port powers corresponding to a plurality of different initial states of charge and a plurality of different amplitudes of the current sequence, and obtain a current amplitude-state of charge-port power curved surface based on the port powers;
fit the current amplitude-state of charge-port power curved surface to a plane equation; and
obtain a current amplitude corresponding to the port power and the state of charge by using the plane equation based on the port power and the state of charge, and update the state of charge based on the current amplitude and the preset time period.

16. The non-transitory computer readable storage medium of claim 15, wherein the initial state information about the battery comprises: a lithium concentration on a surface of an electrode active material, an average lithium concentration of the electrode active material, a lithium concentration of an electrode electrolyte, and an initial temperature of the battery.

17. The non-transitory computer readable storage medium of claim 16, wherein the processor is further configured to:
obtain an ambient temperature sequence of the battery with a constant amplitude, wherein the ambient temperature sequence of the battery comprises an ambient temperature of the battery at each moment within the preset time period,
at a starting moment of the preset time period, update a parameter vector at a current moment based on the lithium concentration of the electrode electrolyte, the average lithium concentration of the electrode active material and the battery temperature at a previous moment:
$$θ ( k + 1 ) = {f}_{θ} ( {c}_{e} ( k ) , {c}_{s , av} ( k ) , {T}_{b} ( k ) )$$
where θ(k+1) represents the parameter vector at the current moment, ƒₑ represents a parameter update function, cₑ(k) represents the lithium concentration of the electrode electrolyte at the previous moment, c_{s,av}(k) represents the average lithium concentration of the electrode active material at the previous moment, and T_{b}(k) represents the battery temperature at the previous moment;
update a reaction current intensity at the current moment based on the lithium concentration of the electrode electrolyte, the lithium concentration on the surface of the electrode active material, the battery temperature, and port current at the previous moment and the parameter vector at the current moment:
$${j}_{n} ( k + 1 ) = {f}_{j} ( {c}_{e} ( k ) , {c}_{s , surf} ( k ) , {T}_{b} ( k ) , I ( k ) , θ ( k + 1 ) )$$
where jₙ(k+1) represents the reaction current intensity at the current moment, ƒⱼ represents a reaction current update function, c_{s,surf}(k) represents the lithium concentration on the surface of the electrode active material at the previous moment, and I(k) represents the port current at the previous moment;
update a potential difference on a solid-solution surface of an electrode at the current moment based on the reaction current intensity and the parameter vector at the current moment:
$${ϕ}_{se} ( k + 1 ) = {f}_{ϕ} ( {j}_{n} ( k + 1 ) , θ ( k + 1 ) )$$
where ϕₛₑ(k+1) represents the potential difference on the solid-solution surface of the electrode at the current moment, and ƒ_{ϕ} represents an update function of the potential difference on the solid-solution surface of the electrode;
update the lithium concentration of the electrode active material at the current moment based on the average lithium concentration of the electrode active material and the lithium concentration on the surface of the electrode active material at the previous moment, the reaction current intensity at the current moment, the parameter vector at the current moment and a sampling interval:
$${c}_{s , av} ( k + 1 ) = {f}_{av} ( {c}_{s , av} ( k ) , {c}_{s , surf} ( k ) , {j}_{n} ( k + 1 ) , θ ( k + 1 ) , Δ t )$$
$${c}_{s , surf} ( k + 1 ) = {f}_{surf} ( {c}_{s , av} ( k ) , {c}_{s , surf} ( k ) , {j}_{n} ( k + 1 ) , θ ( k + 1 ) , Δ t )$$
where c_{s,av}(k+1) represents the average lithium concentration of the electrode active material at the current moment, ƒₐᵥ represents an update function of the average lithium concentration of the electrode active material, Δt represents the sampling interval, c_{s,surf}(k+1) represents the lithium concentration on the surface of the electrode active material at the current moment, and ƒ_{surf} represents an update function of the lithium concentration on the surface of the electrode active material;
update the lithium concentration of the electrode electrolyte at the current moment based on the lithium concentration of the electrode electrolyte and the port current at the previous moment, the parameter vector at the current moment and the sampling interval:
$${c}_{e} ( k + 1 ) = {f}_{e} ( {c}_{e} ( k ) , I ( k ) , θ ( k + 1 ) , Δ t )$$
where cₑ(k+1) represents the lithium concentration of the electrode electrolyte at the current moment, and ƒₑ represents an update function of the lithium concentration of the electrode electrolyte;
obtain a port voltage V of the battery and a potential difference U in the battery at the current moment based on the lithium concentration of the electrode electrolyte, the lithium concentration on the surface of the electrode active material, the reaction current intensity and the parameter vector at the current moment, and the battery temperature and the port current at the previous moment:
$$V ( k + 1 ) = {f}_{V} ( {c}_{e} ( k + 1 ) , {c}_{s , surf} ( k + 1 ) , {j}_{n} ( k + 1 ) , {T}_{b} ( k ) , I ( k ) , θ ( k + 1 ) )$$
$$U ( k + 1 ) = {f}_{U} ( {c}_{e} ( k + 1 ) , {c}_{s , surf} ( k + 1 ) , {j}_{n} ( k + 1 ) , {T}_{b} ( k ) , I ( k ) , θ ( k + 1 ) )$$
where V(k+1) represents the port voltage of the battery at the current moment, ƒ_{V} represents an update function of the port voltage of the battery, U(k+1) represents the potential difference in the battery at the current moment, and ƒ_{U} represents an update function of the potential difference in the battery;
obtain the battery temperature at the current moment based on the port voltage of the battery, the potential difference in the battery, the reaction current intensity and the parameter vector at the current moment, and the battery temperature, ambient temperature, the port current and the sampling interval at the previous moment:
$${T}_{b} ( k + 1 ) = {f}_{T} ( V ( k + 1 ) , U ( k + 1 ) , {j}_{n} ( k + 1 ) , {T}_{b} ( k ) , {T}_{amb} ( k ) , I ( k ) , θ ( k + 1 ) , Δ t )$$
where T_{b}(k+1) represents the battery temperature at the current moment, ƒ_{T} represents an update function of the battery temperature, and T_{amb}(k) represents the ambient temperature at the previous moment; and
repeat the above-mentioned simulation, iteration and update steps, and cyclically updating states at the current moment from states at the previous moment: the parameter vector, the reaction current intensity, the potential difference on the solid-solution surface of the electrode, the lithium concentration of the electrode active material, and the lithium concentration of the electrode electrolyte, and output the port voltage of the battery and the battery temperature based on a state update result until the preset time period ends to obtain the port voltage of the battery at each moment within the preset time period,
wherein the port voltage of the battery at each moment within the preset time period may be represented as:
V=[V₁V₂ . . . Vₖ . . . V_{N}]
where V represents the port voltage of the battery at each moment within the preset time period, Vₖ represents a port voltage of the battery at a k^{th} moment, and N is a total number of moments within the preset time period, wherein N is an integer greater than or equal to 2.

18. The non-transitory computer readable storage medium of claim 17, wherein the processor is further configured to:
obtain an average port voltage within the preset time period based on the port voltage of the battery, and calculate the port power corresponding to the initial state of charge and the amplitude of the current sequence based on the average port voltage and the amplitude of the current sequence;
wherein the average port voltage is represented as:
$$\overline{V} = \frac{{V}_{1} + {V}_{2} + … + {V}_{k} + … + {V}_{N}}{N}$$
where V̅ represents the average port voltage; and
the port power is represented as:
$$P ( {SOC}_{0} , {T}_{amb} ) + I \times \overline{V}$$
where P represents the port power, SOC₀ represents the initial state of charge, T_{amb} represents a set ambient temperature of the battery, and I represents the amplitude of the current sequence.

19. The non-transitory computer readable storage medium of claim 18, wherein the processor is further configured to:
adjust the initial state of charge and the amplitude of the current sequence of the battery, repeat the above steps to respectively obtain port powers corresponding to the plurality of different initial states of charge and the plurality of different amplitudes of the current sequence, and smoothly connect the port powers to obtain the current amplitude-state of charge-port power curved surface under a condition of maintaining the ambient temperature of the battery constant;
wherein the current amplitude-state of charge-port power curved surface is represented as:
$${{f}_{P} ( {SOC}_{0} , P , I ) \vert}_{T = {T}_{amb}} = 0$$
where ƒ_{P} represents a function of the current amplitude-state of charge-port power curved surface in a case of T=T_{amb}, and T represents an actual ambient temperature of the battery.

20. The non-transitory computer readable storage medium of claim 19, wherein fitting the current amplitude-state of charge-port power curved surface to the plane equation is represented as:
$${{f}_{P} ( {SOC}_{0} , P , I ) \vert}_{T = {T}_{amb}} \approx A \times {SOC}_{0} + B \times P + C \times I + D = 0$$
where ƒ_{P} represents the function of the current amplitude-state of charge-port power curved surface in the case of T=T_{amb}, A, B and C are first-order coefficients of plane fitting, and D is a constant coefficient of the plane fitting.
